(19) United States
(12) Patent Application Publication
(10) Pub. No.: US 2012/0108979 A1
(43) Pub. Date: May 3, 2012

(54) IMAGING CATHETER WITH ROTATABLE ARRAY

(75) Inventors: Curtis Franklin, Flagstaff, AZ (US); Thomas W. Shilling, Lakewood, CO (US); Dennis R. Dietz, Littleton, CO (US); Craig T. Nordhausen, Parker, CO (US); Dennis Dreher, Williams, AZ (US); Thomas L. Tolt, Aurora, CO (US)

(73) Assignee: Gore Enterprise Holdings, Inc., Newark, DE (US)

(21) Appl. No.: 13/283,354

(22) Filed: Oct. 27, 2011

Related U.S. Application Data
(60) Provisional application No. 61/407,382, filed on Oct. 27, 2010.

(51) Int. Cl. A61B 8/14

(52) U.S. Cl. 600/463

(57) ABSTRACT

An imaging catheter is provided having a distal end portion selectively rotatable relative to a catheter body. A transducer array is supported by the distal end portion so that a corresponding imaging field may be selectively panned about an axis extending distally from the catheter body. The catheter may be advanced within a patient to a desired location. Optionally, the catheter may then be steered, or curved to position the transducer array. Optionally, the catheter may be rotated to further position the transducer array. Then, the imaging field may be panned without manipulation of the catheter body.

### RELATED APPLICATION

This application claims priority to U.S. Provisional Patent Application No. 61/407,382, filed Oct. 27, 2010, entitled “IMAGING CATHETER WITH ROTATABLE ARRAY,” which application is incorporated herein by reference in its entirety.

### FIELD OF THE INVENTION

The present invention relates to catheters, and more particularly to imaging catheters with enhanced positioning capabilities.

### BACKGROUND OF THE INVENTION

Catheters are medical devices that may be inserted into a body vessel, cavity or duct, and manipulated utilizing a portion that extends out of the body. Typically, catheters are relatively thin and flexible to facilitate advancement/retraction along non-linear paths. Catheters may be employed for a wide variety of purposes, including the internal bodily positioning of diagnostic and/or therapeutic devices. For example, catheters may be employed to position internal imaging devices (e.g., ultrasound transducers).

In this regard, use of ultrasonic imaging techniques to obtain visible images of structures is increasingly common. Broadly stated, an ultrasound transducer, typically comprising a number of individually actuated piezoelectric elements arranged in an array, is provided with suitable drive signals such that a pulse of ultrasonic energy travels into the body of the patient. The ultrasonic energy is reflected at interfaces between structures of varying acoustic impedance. The same or a different transducer detects the receipt of the return energy and provides a corresponding output signal. This signal can be processed in a known manner to yield an image, visible on a display screen, of the interfaces between the structures and hence of the structures themselves.

In one application, Intracardiac Echocardiography (ICE) catheters have become the preferred imaging modality for use in some structural heart interventions because they provide high resolution 2D ultrasound images of the soft tissue structure of the heart. Additionally, ultrasound imaging does not contribute ionizing radiation to the procedure. ICE catheters can be used by the interventional cardiologist and staff within the context of their normal procedural flow and without the addition of other hospital staff. Current ICE catheter technology does have limitations though. Conventional ICE catheters are limited in that the clinician must repeatedly manipulate the catheter in order to capture multiple image planes within the anatomy. The catheter manipulation needed to obtain specific 2D image planes requires that a user spend a significant amount of time becoming facile with the catheter steering mechanisms.

As internal diagnostic and therapeutic procedures continue to evolve, the desirability of enhanced procedure imaging via compact and maneuverable catheters has been recognized by the present inventors. More particularly, the present inventors have recognized the desirability of providing catheter features that facilitate selective positioning of imaging componentry located at a distal end of a catheter, while maintaining a relatively small profile, thereby yielding enhanced functionality for various clinical applications.

As may be appreciated, the utilization of ultrasound transducers on catheters presents dimensional challenges, particularly for vascular applications. For example, for cardiovascular applications it may be desirable to maintain a maximum cross-dimension of less than about 12 French (Fr), and more preferably less than about 10 Fr, during advancement of an imaging catheter into the right atrium or other chambers of the heart.

### SUMMARY OF THE INVENTION

In one embodiment, an imaging catheter is provided that comprises a catheter body and a distal end portion supported by and selectively rotatable relative to a distal end of the catheter body at an interface therebetween. The imaging catheter further includes at least one electrical signal line extending within the catheter body between a proximal end and a distal end thereof, and a transducer array supported by the distal end portion and electrically interconnected to the electrical signal line across the interface. In turn, the transducer array may have a predetermined imaging field that is selectively rotatable about a predetermined axis extending distally away from the distal end of the catheter body. The predefined imaging field may be selectively rotated, or panned, back-and-forth within a predetermined angular range of at least 360°.

In certain implementations, the imaging catheter may be provided so that the interface includes a fluid seal between the distal end of the catheter body and the rotatable distal end portion. In one approach, a seal member may be provided between interfacing surfaces provided on the catheter body and the distal end portion. In another approach, the interface surfaces may cooperate to provide a fluid seal therebetween with or without the inclusion of a seal member.

In some applications, the imaging catheter may be provided so that the interface restricts undesired rotative movement of the distal end portion relative to the distal end of the catheter body. By way of example, a compression force may be applied between the interfacing surfaces at the distal end of the catheter body and the distal end portion, wherein the interfacing surfaces may move relative to one another upon the application of a predetermined minimum force (e.g., applied by a user), and wherein frictional resistance attendant to the compression force may restrict such relative movement in the absence of the application of the predetermined minimum force.

In some embodiments, the imaging catheter may be provided so that the interface includes at least a first bearing surface at the distal end of the catheter body and at least a second bearing surface on the distal end portion. The first bearing surface and the second bearing surface may be disposed in opposing contact relation to provide a fluid seal and/or resistance to undesired rotative movement of the distal end portion relative to the distal end of the catheter body.

In various embodiments, at least one of the first bearing surface and the second bearing surface may be elastically deformed. In that regard, the first and/or second bearing surface(s) may be radially and/or axially, elastically deformed to provide a fluid seal and/or resistance to undesired rotative movement of the distal end portion relative to the distal end of the catheter body. To provide such functionality, at least one of the bearing surfaces may comprise an elastomeric material, a thermoplastic elastomeric material, a thermoplastic material, or another elastically deformable material. By way of example, one or more elastomeric O-ring(s) or sleeves may be utilized and/or an overmolded, thermoplastic, elastomeric or thermoplastic elastomeric layer. In another approach, at least one of the first and second bearing surfaces may be presented as a spring-loaded component.

Opposing portions of a first bearing surface and a second bearing surface may be provided to have coincidental configurations extending about a longitudinal axis of the imaging catheter. For example, the opposing surface portions may be of coincidental annular configurations extending about and/or along a longitudinal axis of the imaging catheter (e.g., a center axis). Further, the opposing portions of the first bearing surface and second bearing surface may be provided so that all or substantially all contact therebetween along a longitudinal axis of the catheter body (e.g., a center axis) is within a ring-shaped portion (e.g., a donut-shaped portion) having an inner radius and outer radius (e.g., relative to the longitudinal axis) that corresponds with about 40% or less of the outer radius of the imaging catheter, thereby providing an inner cylindrical portion having a radius that corresponds with about at least 60% of the outer radius of the imaging catheter. As may be appreciated, the cylindrical inner volume facilitates the passage of other componentry therethrough. In one arrangement, the first and second bearing surfaces may be provided to that all or substantially all contact therebetween along a longitudinal axis of the catheter body (e.g., a center axis) is substantially equidistance from the longitudinal axis or within a predetermined range of radial distance variance relative to the longitudinal axis (e.g., a radial distance variance that represents about 40% or less relative to the outside radius of the imaging catheter).

In one arrangement, an imaging catheter may be provided having an interface that includes a spaced plurality of first bearing surfaces at the distal end of the catheter body and at least one second bearing surface or a spaced plurality of second bearing surfaces on the distal end portion. The plurality of first bearing surfaces may be disposed in opposing contact relation with the at least one first bearing surface on the distal end portion to provide a fluid seal between the catheter body and the distal end portion. In this regard, one or more of the bearing surfaces may be elastically deformed as described above. Further, the bearing surfaces may be configured with coincidental configuration features as described above.

As may be appreciated, the imaging catheter may include a force communication member operable to apply a force to rotate the distal end portion relative to the distal end of the catheter body. In one approach, the force communication member may include a drive member extending through the catheter body (e.g., through a tubular passageway thereof) from the proximal end to the distal end thereof. The drive member may be fixedly interconnected to the distal end portion, wherein a proximal end of the drive member may be selectively rotated to affect selective rotation of the distal end portion and the transducer array. In turn, panning of the predetermined imaging field across a predetermined angular range may be realized.

In one implementation, the drive member may comprise a shaft extending through the catheter body. In such implementation, the electrical signal may be provided to extend along and about the shaft to the distal end portion, e.g., the electrical signal line may helically wrap about the shaft. In another implementation, the drive member may comprise a tubular member extending through the catheter body. In such implementation, the electrical signal line may be routed through the tubular member to the distal end portion, e.g., the electrical signal line may helically extend within the tubular member.

In another approach, mechanical force may be communicated from a proximal end of the catheter to the distal end portion via longitudinal advancement/retraction of one or more members along the length of the catheter body. For example, a pair of flexible elongate members (e.g., wires) may be interconnected at their distal ends to a support member for the transducer array and separately retracted (e.g., pulled) to rotate the transducer array in a desired direction about a predetermined axis, e.g., wherein proximal retraction of a first wire effects distal advancement of a second wire. In one embodiment, first and second wires may be operatively interconnected to a spool member in the distal end portion to selectively rotate the spool member and thereby effect rotation of the transducer array.

In yet other approaches, the force communication member may utilize hydraulic, pneumatic, magnetic and/or electrical componentry to provide for selective rotation of the distal end portion. In each of such arrangements, the actuation, or initiation, of the application of the rotative force may be initiated via operator control at a proximal end of the catheter.

Optionally, the imaging catheter may incorporate various approaches to provide for steering of a catheter body. For example, one or a plurality of pull wires may extend from a proximal end to a distal end portion of a catheter body. The distal ends of the pull wire(s) may be anchored in a distal portion of the catheter body, wherein application of a tensile force to a pull wire may affect the flexure, or curvature, of the catheter body in a direction corresponding with the relative position of the pull wire within the catheter body.

In one implementation, the catheter may be provided with a plurality of segments having different stiffnesses. For example, a first segment may have a first stiffness, and a second segment disposed distal to the first segment may have a second stiffness, wherein the first stiffness is greater than the second stiffness. In turn, the second segment may be deformable to a smaller radius of curvature than that of the first segment in response to a given tensile force applied by a pull wire. In some implementations, the first segment may be provided to be deformable to a first radius of the curvature (R₁) and the second segment may be provided to be deformable to a second radius of curvature (R₂) in response to a tensile force applied by a pull wire, wherein a ratio of R₂/R₁ is no more than about 2/3 and in certain implementations no more than about 1/2. The second segment may comprise a distal end of the catheter body, wherein the second segment is deformable to a second radius of curvature of about 10 cm or less, and in some implementations 4 cm or less or even 2 cm or less.

In some embodiments, the catheter may be deformable to a predetermined minimum radius of curvature along an entire length thereof in response to a tensile force applied to a pull wire. In turn, the electrical signal line may include an electrical signal member of a ribbon-like configuration (e.g., having a plurality of electrically conductive members supported on a support layer that may include a ground layer) that extends helically about the center axis of the catheter body at a predetermined wrap angle within a range established so that the electrical signal line maintains a non-overlapping disposition (e.g., the electrical signal member does not have portions that overlap) when the catheter body is deformed to the predetermined minimum radius of curvature. By way of example, such predetermined wrap angle range may be about 10° to 80°, and in certain arrangements about 20° to 45°.

In some arrangements, the electrical signal line may include at least one electrical signal member of ribbon-like configuration that extends helically about a center axis of the catheter body (e.g., along a length of the catheter body), wherein at least a section of the length of the electrical signal member is provided to tighten/loosen (e.g., wind/unwind) in conjunction with selective rotation of the distal end portion away from/toward a “home” position. By way of example, the section may be free from fixed interconnection with other componentry between distal and proximal ends of such section. In certain implementations where the catheter may be provided with a plurality of segments having different stiffnesses (e.g., a first segment having a first stiffness greater than a second stiffness of a second segment), the referenced section of the electrical signal member section may be provided to extend through all or at least a portion of the second segment so as to accommodate deformation of the second segment during steering.

In some embodiments a plurality of electrical signal members may extend through the catheter body from the proximal end thereof. For example, a plurality of ribbon-like electrical support members may helically extend through the catheter body in adjacent (e.g., alternating) and/or stacked relation.

As may be appreciated, the selectively rotatable transducer array may provide an output signal that may be processed to yield two-dimensional (2D) images. Further, in some implementations the output signal may be processed to provide three-dimensional (3D) images. For example, in one approach the output signal may be processed together with corresponding information indicative of the rotation, or position, of the transducer array to yield 3D images. In one implementation, a positional encoder may be utilized to provide a position signal (e.g., indicative of the position of the transducer array) employable with the transducer array output signal. In other approaches, the transducer array may be rotated to a given position, then separately reciprocated relative to the distal end portion, e.g., utilizing an actuator or motor drive.

A method is also provided for imaging a predetermined region of interest within a patient body. In one embodiment, the method may include the steps of advancing a catheter within a patient body, wherein the catheter includes a catheter body with a distal end portion supported at a distal end of the catheter body. After such advancement, the method may further include the step of rotating the distal end portion relative to the distal end of the catheter body. In this regard, the rotating step may be completed free from manipulation of the catheter body (e.g., free from further advancement).

The method may further include the step of obtaining an output signal from a transducer array supported by the distal end portion after and/or during at least a portion of the rotating step. In this regard, the transducer array may have a predetermined imaging field that is positionable at a plurality of locations. In turn, the method may provide for processing of an output signal to obtain image data corresponding with the plurality of locations. Such image data may be utilized to generate images displayed to a user during a diagnostic or therapeutic procedure.

In some embodiments, the output signal may be processed to yield 2D image data. Further, in some applications the output signal may be processed to yield 3D image data. For example, 2D image data may be processed together with information indicative of corresponding transducer array positioning to provide 3D image data.

In some implementations, the distal end portion and the catheter body of the catheter may comprise at least a first bearing surface and at least a second bearing surface, respectively. In turn, the rotating step may include moving the first bearing surface relative to and in contact engagement with the second bearing surface. In certain implementations, the method may further include maintaining a fluid seal between a distal end portion and a distal end of the catheter body throughout the moving step. By way of example, a seal member may be employed with the first bearing surface and the second bearing surface. In another approach, the first and second bearing surfaces may be defined to provide the fluid seal without the utilization of a separate seal member. For example, one of the first and second bearing surfaces may be elastically deformed so as to provide a fluid seal. In one approach, one of such bearing surfaces may comprise a thermoplastic, a thermoplastic elastomeric, or an elastomeric material (e.g., one or more elastomeric O-rings and/or an overmolded, thermoplastic or thermoplastic elastomeric surface layer).

In some embodiments, the first bearing surface and second bearing surface may be provided to resist relative movement therebetween in the absence of the applications of predetermined minimum amount of force to the distal end portion. By way of example, the first and second bearing surfaces may be provided with a compression interface therebetween. The compression interface may be provided radially and/or axially relative to a longitudinal axis of the imaging catheter. Such compression interface may be established to yield frictional resistance to relative movement therebetween in the absence of the application of the predetermined minimum amount of force to the distal end portion.

In this regard, in one embodiment the catheter may include a drive member interconnected to the distal end portion and extending from the proximal end of the catheter body to a distal end thereof. In turn, the rotating step may include manipulating the drive member at the proximal end of the catheter body to apply at least the predetermined amount of force necessary to affect selective relative rotation of the distal end portion.

In one method embodiment, a first length of the catheter may be advanced into a patient wherein the distal end portion may be located in a first position. Then, the catheter body may be steered to curve a distal end portion of the catheter body (e.g., without additional advancement of the catheter into the patient), wherein the distal end portion may be located in a second position.

Next, the catheter may be rotated at the proximal end, or twisted, to effect rotation along the length thereof, wherein the distal end portion may be located in a third position (e.g., without additional advancement of the catheter into the patient). Finally, the distal end portion may be rotated relative to the distal end of the catheter body to yield an image signal for processing (e.g., without manipulation of the catheter body). The noted four steps may be carried in any order, and repeated, any number of times.

As may be appreciated, the present invention is of particularly apt for catheter applications which may benefit from the capability to move the transducer array independent of a catheter body. Of particular benefit is the capability that allows a transducer array to be rotated relative to an axis (e.g., a center axis) of the catheter extending distally from a distal end of the catheter, wherein a panning motion may be realized. This panning motion provides the capability to rotate the transducer array in a 2D ICE catheter to capture multiple imaging views from a single catheter body position. As noted, this same panning capability may also be beneficial in a 3D catheter with a wobble mechanism interfaced with the transducer. In the case of the 3D catheter, the wobble mechanism may generate the 3D scan volume by oscillating the transducer across an imaging field and the panning motion allows for selection of the center point of the transducer scan volume.

Using such a catheter-based imaging system for visualizing the three dimensional (3D) architecture of the heart, for example, on a real-time basis during intervention may be highly desirable from a clinical perspective as it may facilitate more complex procedures such as left atrial appendage occlusion, mitral valve repair, and ablation for atrial fibrillation. 3D imaging may also allow the clinician to fully determine the relative position of structures. This capability may be of particular import in cases of structural abnormalities in the heart where typical anatomy is not present. Two-dimensional transducer arrays provide another means to generate 3D images, and similar panning motion at the catheter distal end containing the transducer array may be applied to select the center point of the 3D scan volume. Currently available 2D arrays require a high number of elements in order to provide sufficient aperture size and corresponding image resolution. In turn, the high element count may result in a 2D transducer array that is prohibitive with respect to clinically acceptable catheter profiles. On the other hand, 2D transducers with the panning capability disclosed herein may be well-suited for use numerous applications.

Numerous additional features and advantages of the present invention will become apparent to those skilled in the art upon consideration of the embodiment descriptions provided hereinbelow.

### BRIEF DESCRIPTION OF THE DRAWINGS

FIG. 1 illustrates one embodiment of an imaging catheter distal end portion.

FIG. 2 is a cross-sectional view of a distal end portion and catheter body portion of the embodiment of FIG. 1.

FIG. 3 is a cross-sectional view of interface members of the embodiment of FIGS. 1 and 2.

FIG. 4 is a cross-sectional view of an alternative embodiment of interface members employable in the catheter embodiment of FIGS. 1 and 2.

FIG. 5 is a cross-sectional view of an imaging catheter embodiment.

FIG. 6 illustrates a proximal end of an imaging catheter embodiment.

FIGS. 7A and 7B are cross-sectional views of a distal end portion of an imaging catheter embodiment.

FIGS. 8A and 8B are perspective views of an alternate catheter body embodiment employable in the embodiment shown in FIG. 1.

FIG. 8C is a cross-sectional view of the catheter embodiment shown in FIGS. 8A and 8B.

FIG. 9 illustrates a distal end of another imaging catheter embodiment.

FIG. 10 illustrates an exploded view of a bearing assembly embodiment disposed relative to a distal end of a catheter body.

FIG. 11 is a cross-sectional view of the bearing assembly embodiment of FIG. 10.

FIG. 12 is a cross-sectional view of a portion of the bearing assembly embodiment of FIG. 10 secured to a catheter body.

FIG. 13 is a perspective view of an embodiment of a distal end portion of a drive member support structure, showing first and second electronical signal members and a transducer array electrically interconnected.

FIG. 14 is a perspective view of a portion of an electrical signal line embodiment in an assembled configuration.

FIG. 15 is a detailed view of a drive member support structure.

FIG. 16 illustrates a perspective view of an embodiment of an imaging catheter during a stage of assembly thereof.

FIG. 17 illustrates a perspective view of an embodiment of an imaging catheter during a stage of assembly thereof.

FIG. 18 illustrates a perspective view of an embodiment of an imaging catheter during a stage of assembly thereof.

FIG. 19 illustrates a perspective view of an embodiment of a drive member engaged with a housing assembly.

FIG. 20 illustrates a perspective view of an embodiment of an imaging catheter during another stage of assembly thereof.

FIG. 21 illustrates an embodiment of a housing assembly during a stage of assembly.

FIG. 22 illustrates a cross sectional view of a distal end of an embodiment of an imaging catheter.

### DETAILED DESCRIPTION

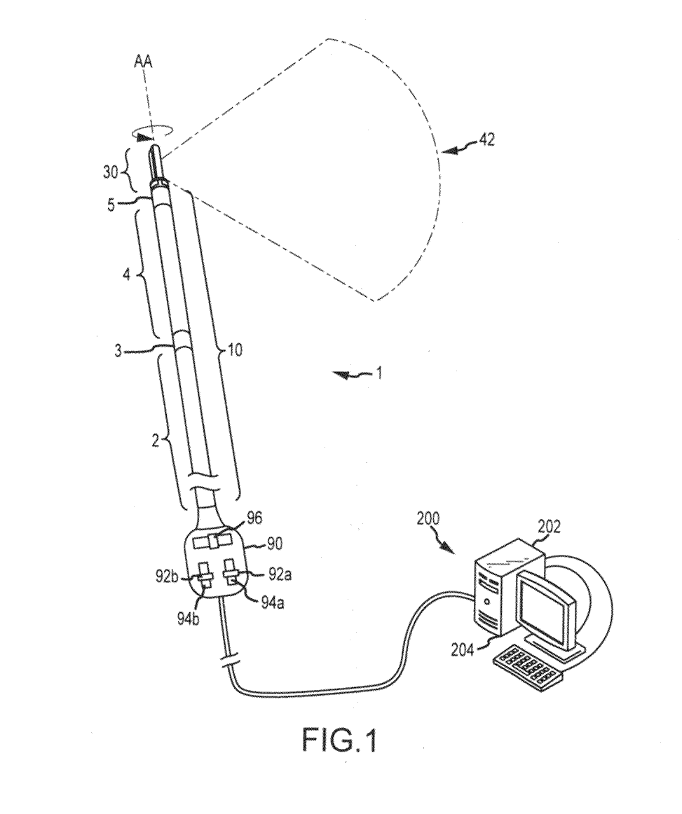

FIGS. 1-3 illustrate one embodiment of an imaging catheter 1. The imaging catheter 1 may include a catheter body 10 and a distal end portion 30 supported by and selectively rotatable relative to a distal end of the catheter body 10 at an interface therebetween.

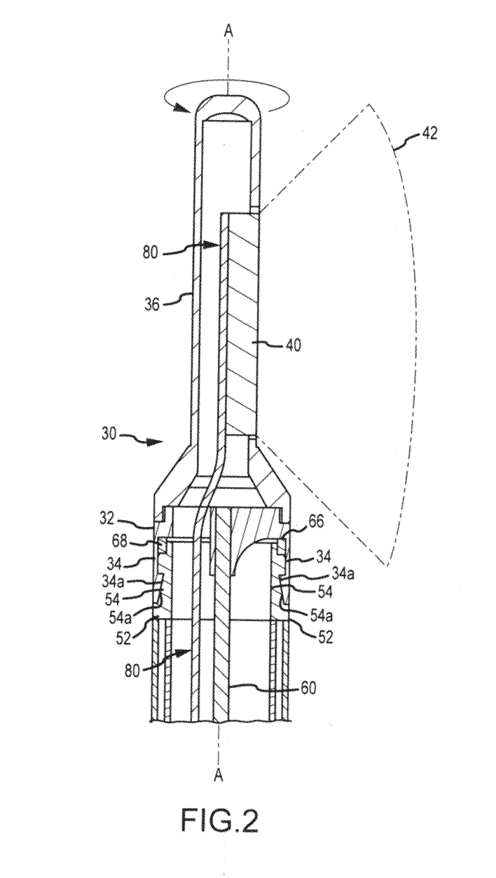

As shown in FIG. 2, the distal end portion 30 of the catheter 1 may include a first interface member 32 and a housing member 36 supportably interconnected to the first interface member 32. In turn, a transducer array 40 (e.g., an ultrasound transducer array) may be rotatably supported by the distal end portion 30. The transducer array 40 may be provided to have a predetermined imaging field 42. The predetermined imaging field 42 may be selectively rotated about axis AA. In the illustrated embodiment, axis AA coincides with a central axis of the distal end portion 30 and a central axis of the catheter body 10.

To facilitate the selective rotation of the distal end portion 30, a rotatable drive member 60 may be disposed through the catheter body 10 and fixedly and sealably interconnected to the first interface member 32. In turn, upon selective rotation of drive member 60 the distal end portion 30, together with transducer array 40, may be rotated for panning the predetermined imaging field 42 across a predetermined angular range. The predetermined angular range may extend up to 360°, and in the illustrated embodiment may be readily established between about 90° to 180°.

As may be appreciated, such a predetermined angular range allows a user to position the catheter at a given location at which the distal end portion may be selectively rotated to pan the imaging field 42 and advantageously view desired bodily structures within the predetermined angular range, free from manipulation of the catheter body 10. Such an approach reduces unpredictable movement of an imaging field that may occur in certain prior art arrangements that entail manipulation of a catheter body in order to reposition an imaging field.

In some implementations, the predetermined imaging field 42 may be selectively panned in a first direction about axis AA within said predetermined angular range, then selectively panned in a second direction opposite to the first direction within the predetermined angular range. In some embodiments, such back-and-forth panning may be readily repeated as desired by medical personnel.

The interface between the distal end portion 30 and catheter body 10 may be defined by the first interface member 32 and a second interface member 52 provided at the distal end of the catheter body 10. In one approach, the first interface member 32 may include a lateral portion 34 having a bearing surface 34a, and the second interface member 52 may include an adjacent lateral portion 54 having a bearing surface 54a. The first bearing surface 34a and second bearing surface 54a may be sized and/or otherwise configured to facilitate rotation of first bearing surface 34a relative to the second bearing surface 54a while maintaining contact engagement therebetween.

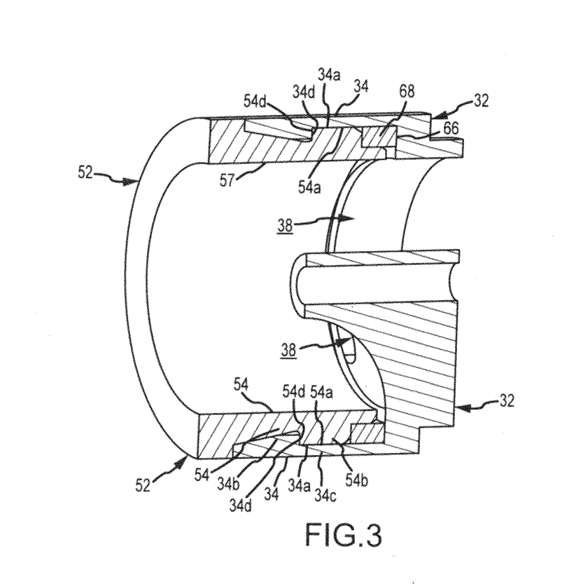

As shown in FIG. 3, the lateral portion 34 of the first interface member 32 may be sized to matingly receive at least a portion of the lateral portion 54 of the second interface member 52 therewithin. By way of example, the lateral portion 34 may comprise an enlarged head 34b and reduced neck 34c. Similarly, the catheter portion 54, may comprise an enlarged head 54b and reduced neck 54c. The enlarged head 34b and reduced neck 54c may be of complimentary configurations and/or the enlarged head 54b and reduced neck 34c may be of complimentary configurations for contact engagement therebetween. Further, such configuration(s) may facilitate snap-fit interconnection and simplified assembly.

The two lateral portions 34, 54 may be sized so as to define a slot region 66 adjacent to a distal end of the second interface member 52. As shown in FIG. 2 and FIG. 3, an annular seal member 68 (e.g., an O-ring) may be disposed within the slot region 66. In turn, the seal member 68 may provide a sealed interface between the catheter body 10 and the rotatable distal end portion 30. As may be appreciated, seal member 68 may be sized so as to be in axial and/or radial compression upon assembly.

In some arrangements, the first bearing surface 34a of the first interface member 32 and the second bearing surface 54a of the second interface member 52 may be sized and/or configured to allow for rotation of the first bearing surface 34a relative to second bearing surface 54a, while also providing sufficient frictional resistance to relative rotation in the absence of the application of a predetermined amount of force by the drive member 60. In this regard, a compression interface between first bearing surface 34a and second bearing surface 54a may be provided that is sufficient to frictionally restrict unintended relative movement between the first interface member 32 and the second interface member 52, e.g., in response to rotative positioning of the distal end portion using drive member 60. By way of example, abutting ledge surfaces 34d and 54d may engage with a compression force therebetween (e.g., as a result of a snap-fit arrangement).

In one embodiment, the first bearing surface 34a and second bearing surface 54a may be provided to define a compression interface that may also provide a fluid seal, free the inclusion of a separate seal member.

Referring again to FIG. 2, the imaging catheter 1 may include an electrical signal line 80 extending between a distal end and proximal end thereof. In this regard, the electrical signal line 80 may include at least a first electrical signal member 82 that extends from a distal end portion of the catheter body 10 and electronically connects to the transducer array 40 across the noted interface. By way of example, the first electrical signal member(s) 82 may comprise one or more flex board circuit members comprising a flexible substrate and a plurality of electrically conductive members supported thereupon (e.g., metal traces). As may be appreciated, the utilization of flexible first electrical signal member(s) 82 facilitates relative rotational movement of the transducer array 40 relative to the catheter body 10.

Further in that regard, reference is again made to FIG. 3. As shown, the first interface member 32 may comprise a slot 38 (e.g., an arcuate slot). In turn, the flexible electrical signal member(s) 82 may be positioned through the slot 38, wherein the slot 38 moves back-and-forth about the electrical signal member(s) 82 during rotative movement of the distal end portion 30.

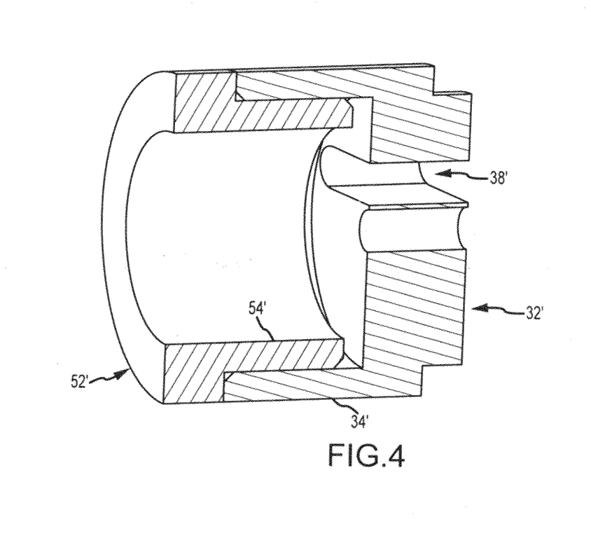

FIG. 4 illustrates a portion of the catheter 1 embodiment with modified versions of a first interface member 32′ and a second interface member 52′. In this arrangement, the lateral portion 34′ of the first interface member 32′, and the lateral portion 54′ of the second interface member 52′ may be of simple, planar configurations. Again, a slot 38′ may be provided for passage of an electrical signal line 80 therethrough (e.g., an oblong slot that may rotate with electrical signal member(s) 82).

Reference is again made to FIG. 1. The catheter 1 may comprise a plurality of segments 2, 3, 4 and 5 along the length of the catheter body 10. The catheter 1 may be provided so that the stiffness of different ones of the plurality of such segments may be different so as to provide desired steerability. For example, the stiffness of catheter 1 may decrease from the proximal end to the distal end of the catheter body 10.

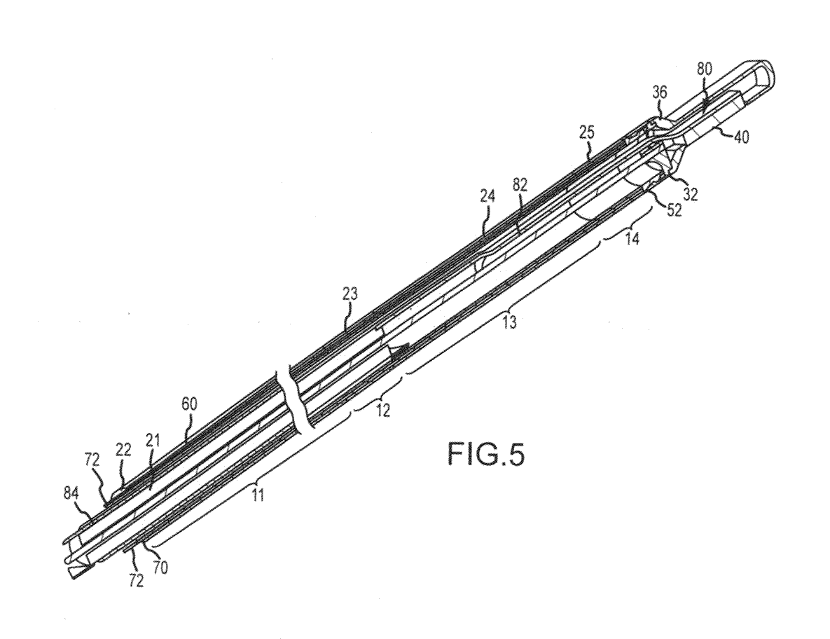

By way of example, and referring now to FIG. 5, a first segment 2 of the catheter 1 may correspond with a first portion 11 of the catheter body 10 comprising a tubular inner member 21 and a tubular outer member 22. In one implementation, the inner member 21 and/or outer member 22 may be extruded utilizing a polymer-based material, e.g., a polyether block amide (PEBA) such as PEBAX™. In one approach the outer member 22 and/or inner member may be extruded from PEBAX to yield a durometer hardness of about 63 to 82, e.g., about 72.

The tubular inner member 21 may be sized to receive the drive member 60 therethrough, wherein the drive member 60 may be rotated relative to the inner tubular member 21. Further, the inner tubular member 21 and outer tubular member 22 may be sized to accommodate the passage of an electrical signal line 80 in the form of a second electrical signal member 84 therebetween, as further described below.

The tubular outer member 22 or tubular inner member 21 may be provided with one or more passageways, or channels, extending therethrough to facilitate the passage of a pull wire for steering catheter body. In the embodiment shown in FIG. 5, the tubular outer member 22 is provided with a plurality of passageways 70, or channels, extending therethrough to facilitate the passage of corresponding pull wires 72 for steering the catheter body 10. Such pull wires 72 may extend from a proximal end of catheter body 10 and may be anchored in a distal end portion of the catheter body 10, wherein the catheter body 10 may be curved, i.e., steered, in a desired direction via the application of a tensile force to one or more of the pull wires.

In this regard, a second segment 3 of the catheter 1, corresponding with a second portion 12 of the catheter body 10, may be provided to have a stiffness that may be less than the first segment 2, wherein the second segment 3 may flex, or curve, to a somewhat greater extent than the first segment 2 in response to a tensile force applied to one or more of the pull wires 72. For such purposes, the second portion 12 may include an outer tubular section 23, as opposed to the outer tubular member 22 of first portion 11, disposed about inner tubular member 21. The outer tubular section 23 may have a corresponding stiffness that may be less than the stiffness of the outer tubular member 22 present in the first portion 11. By way of example, outer tubular section 23 may be extruded utilizing a polymer-based material, e.g., a polyether block amide (PEBA) such as PEBAX™. In one approach the outer tubular section 23 may be extruded from PEBAX to yield a durometer hardness of about 52 to 72, e.g., about 63.

Further, a third segment 4 of the catheter, corresponding with a third portion 13 of the catheter body 10, may have a stiffness that may be less than the stiffness of the second portion 12 and the first portion 11. In the illustrated embodiment, the third portion 13 includes an outer tubular section 24. Such outer tubular section 24 may be of a stiffness that may be less than the combined stiffness of the inner tubular member 21 and outer tubular member 22 comprising the first portion 11 of the catheter body 10, and less than the combined stiffness of the inner tubular member 21 and outer tubular section 23 of the second portion 12. By way of example, outer tubular section 24 may be extruded utilizing a polymer-based material, e.g., a polyether block amide (PEBA) such as PEBAX™. In one approach the outer tubular section 24 may be extruded from PEBAX to yield a durometer hardness of about 35 to 54, e.g., about 40.

Additionally, a fourth segment 5 of the catheter, corresponding with a fourth portion 14 of the catheter body 10 may have a stiffness that may be greater than the stiffness of the third portion 13 and/or second portion 12. In the illustrated embodiment, the fourth portion 14 includes an outer tubular section 25. The greater stiffness may be provided to facilitate anchoring of the pull wires 72 in the fourth portion 14 and steering response to pull wires 72. The outer tubular section 25 may be of a stiffness that may be greater than the stiffness of the outer tubular section 24 of the third portion 13. By way of example, outer tubular section 25 may be extruded utilizing a polymer-based material, e.g., a polyether block amide (PEBA) such as PEBAX™. In one approach the outer tubular section 25 may be extruded from PEBAX to yield a durometer hardness of about 40 to 63, e.g., about 55.

In the described embodiment the second segment 3 functions as a transition between first segment 2 and third segment 4. Also, fourth segment 5 provides for anchoring pull wires 72. In turn, primary steering, or curvature, may be realized in the third segment 4. Utilization of materials as described above facilitates bonding between the various segments 2, 3, 4 and 5.

While not shown, the catheter body 10 may comprise a braided mesh extending along tubular componentry of one or more of the segments 2, 3, 4 and/or 5. For example, a braided mesh may extend under or within the outer tubular sections (e.g., the outer tubular sections may be heated to flow thermoplastic material into the braided mesh). In that regard, in some embodiments, a braided mesh may be provided in which the braid pitch and/or braided elements (e.g., diameter and/or material) may be provided so as vary in stiffness (e.g., decrease in stiffness) along the length of the catheter body 10. That is, a catheter 1 may be provided so that the stiffness of different ones of the segments 2, 3, 4 and/or 5 may be different so as to provide desired steerability. For example, the stiffness of catheter 1 may decrease from the proximal end to the distal end of the catheter body 10.

In one implementation, the first segment 2 of catheter 1 may be deformable to a first radius of curvature (R₁) in response to a tensile force applied by any one of the plurality of pull wires. Correspondingly, the third segment 4 of the catheter 1 may be provided to be deformable to a second radius of curvature (arch 2) in response to such tensile force. The segments 2 and 4 may be provided so that a ratio of R₂/R₁ is no more than about 2/3, and in certain applications no more than about 1/2. As may be appreciated, such an arrangement facilitates steering of catheter 1.

In one embodiment, the stiffness along a length of the catheter 1 may be adjustable. By way of example, the inner tubular member 21 may be provided to be selectively advanceable/retractable within the catheter body 10 (e.g., relative to outer tubular member 22 and outer tubular section 23 and/or outer tubular section 24). In turn, where a greater degree of steering, or relative curvature, is desired more distally, the inner tubular member 21 may be selectively advanced so as to yield greater stiffness along a greater proximal portion. Conversely, where more curvature is desired along a longer proximal length of the catheter, the inner tubular member 21 may be retracted.

As noted above, a second electrical signal member 84 may pass between the inner tubular member 21 and outer tubular member 22 of the first portion 11 of the catheter body 10. Such second electrical signal member 84 may also pass between inner tubular member 21 and outer tubular section 23 of the second portion 12, and within the outer tubular section 24 and outer tubular section 25 of the third portion 13 and fourth portion 14 of the catheter body 10, respectively. The second electrical signal 84 may be helically disposed within the catheter 1 as it extends from the proximal end to the distal end thereof, see, e.g., the third portion 13 and fourth portion 14 of catheter body 10 in FIG. 5.

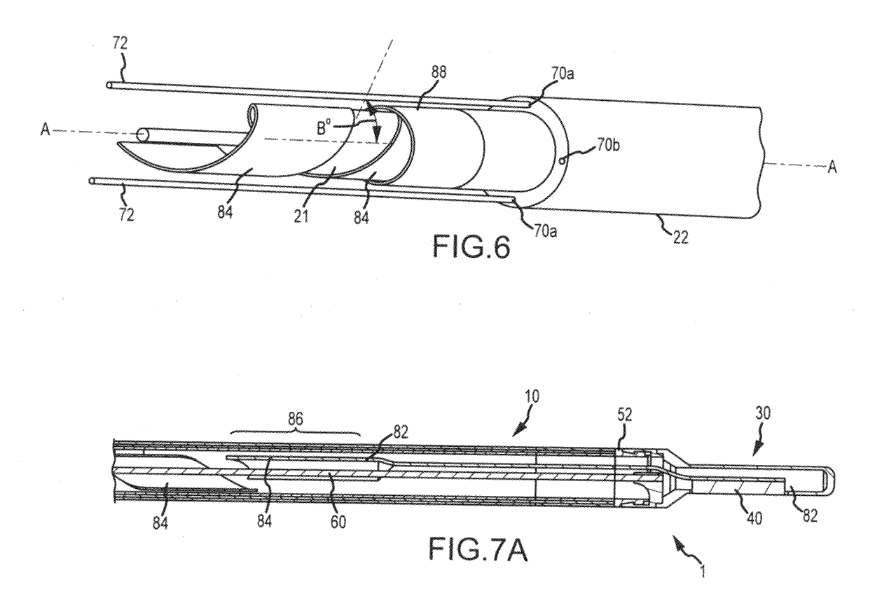

In this regard, reference is now made to FIG. 6 which illustrates componentry of catheter 1 at a proximal end of the catheter body 10. As illustrated, the second electrical signal member 84 may be of a ribbon-like configuration. By way of example, the second electric signal member 84 may comprise a Microflat™ product marketed by W.L. Gore & Associates. As illustrated, the second electric signal member 84 may be helically disposed about a center axis AA of the catheter body 10. Further, in the first portion 12 and the second portion 13 of the catheter body 10, the second electrical signal member 84 may be loosely wound about the inner tubular member 21 facilitate flexure, or curvature, of the catheter body 10. In turn, second electrical signal member 84 may tighten and loosen along inner tubular member 21 as catheter body 10 is steerably advanced during positioning within a patient. In one approach, the second electric signal member 84 may be wound at a wrap angle B of between about 10° to 80° relative to the center axis AA, and in certain implementations between about 20° and 45°.

FIG. 6 further shows a shielding layer 88 that may be provided over the second electrical signal member 84. The shielding layer 88 may shield against electromagnetic interference (EMI). By way of example, shielding layer 88 may extend the length of catheter body 10 and may comprise various conductive foils and/or twisted or braided wires. FIG. 6 also illustrates exemplary pull wires 72 extending through slots 70a. As may be appreciated, additional pull wires may be disposed through slots 70b. As noted, pull wires 72 may be anchored in catheter body portion 14.

In one arrangement, pull wires 72 and drive member 60 may each extend into a handle 90, as shown in FIG. 1. In turn, handle 90 may include slide members 92a and 92b that may be interconnected to different ones of the pull wires 72. The slide members 92a, 92b may be separately advanced and retracted along slide bars 94a, 94b by a user to steer the catheter 1. In one arrangement, each of the slide members 92a, 92b may be rotated laterally for cam-locking an interconnected pull wire in a selected position. Further, handle 90 may include a knob 96 interconnected to drive member 60 for selective user rotation of drive member 60 and distal end portion 30. As noted above, the interface between catheter body 10 and distal end portion 30 may resist relative movement therebetween in the absence of a predetermined timed amount of force applied by a user to knob 96. Such distal end “braking” provided by the described interface facilitates user manipulation of catheter 1 via handle 90.

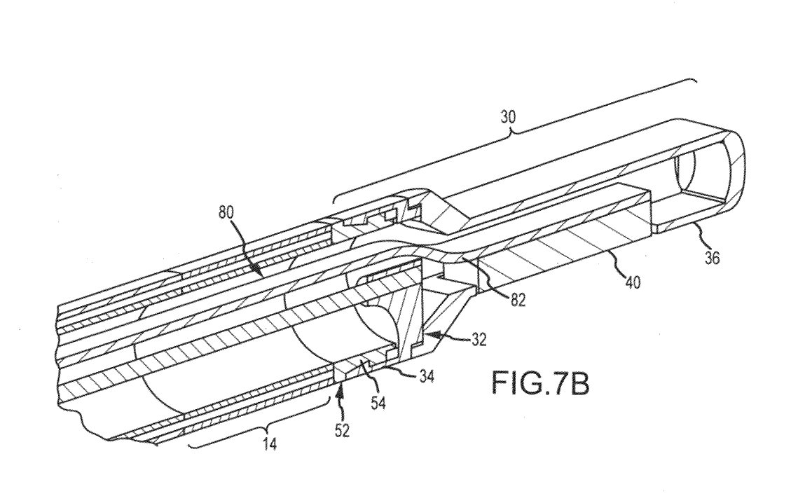

FIGS. 7A and 7B illustrate a distal end portion of the catheter 1, wherein the second electric signal member 84 is shown at an adjoinment region 86 with the first electrical signal member 82 described hereinabove. In the illustrated embodiment, such adjoinment region 86 is provided in the third portion 13 of the catheter body 10.

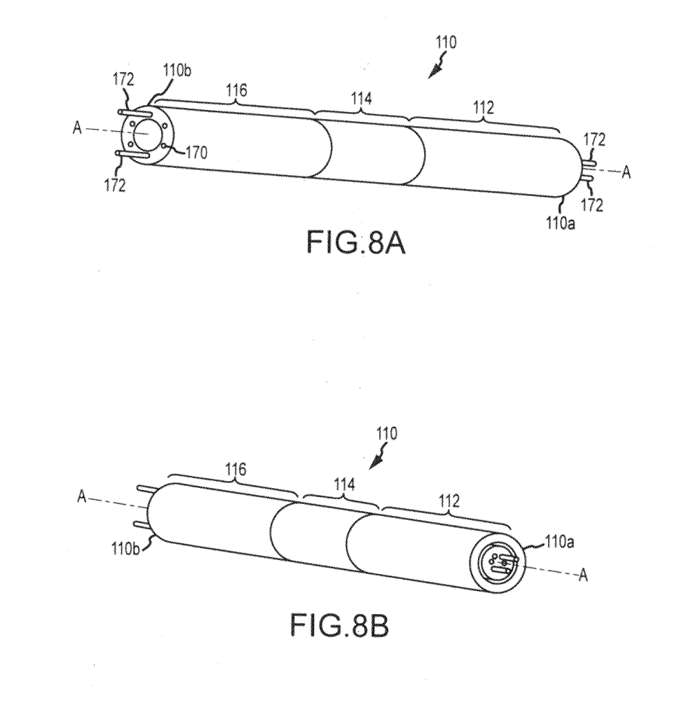

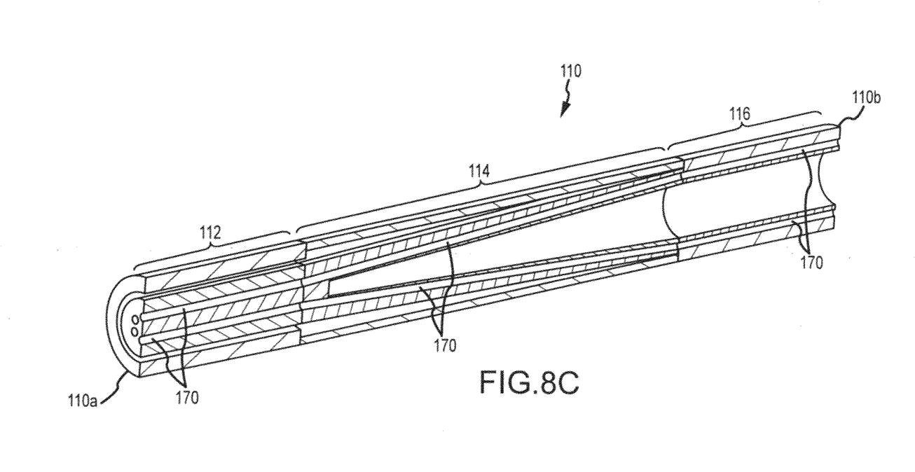

FIGS. 8A, 8B and 8C illustrate a modified catheter body 110 including three portions 112, 114 and 116. Each of the portions 112, 114 and 116 include slots 170 for receiving two or more pull wires 172 therethrough. The slots 170 and pull wires 172 extend away from a center axis AA of the catheter body 110 from a proximal end 110a to a distal end 110b of the catheter body 110. The pull wires 170 are anchored in a distal end portion of the corresponding catheter, wherein an increased moment arm is realized at the distal end of the catheter relative to a proximal end thereof in response to tension applied by the pull wires 170. Such an approach facilitates increased curvature at a distal end portion of the catheter body portion 110 in response to a tensile force applied to one or more pull wires 170, while allowing a catheter body 110 to maintain a relatively constant stiffness along the length thereof.

In certain embodiments the drive member 60 may comprise a shaft. In a modified arrangement, drive member 60 may comprise a tubular member. In turn, the electrical signal line 80 may extend through the tubular member and electrically interconnect to the transducer array 40. In such approach, sealing considerations relating to the interface between the first interface member 32 and second interface member 52 may be reduced, or eliminated.

Returning now to FIG. 1, catheter 1 is shown interconnected to an ultrasound imaging system 200. The system 200 may include an image computer processor 202, operable to process imaging signals from the ultrasound transducer array 40, and an interconnected display device 204, such as a monitor. Imaging signals may be is processed by image processor and the processed image data may be displayed to a user at monitor 204 during a diagnostic and/or therapeutic procedure.

In one contemplated approach utilizing catheter 1, the catheter 1 may be inserted into a patient and advanced to position the distal end portion 30 in a first position. For example, handle 90 may be utilized to advance, e.g., via a pushing motion, a first length of catheter 1 to a first advanced position. Then, the catheter body may be flexed, or steered, to curve the catheter body to a desired curvature, thereby positioning distal end portion 30 in a second position. For example, slide members 92a and 92b may be utilized to control pull wires 72 (e.g., apply a tensile force) and to lock pull wires 72 into a set state, e.g., thereby curving portion 13 of catheter body 10 to a desired curvature. Next, the catheter 1 may be rotated, or twisted (e.g., torqued), to position the distal end portion 30 in a third position. For example, handle 90 may be turned to rotate catheter 1 along the length thereof. Then, distal end portion 30 may be selectively rotated utilizing drive member 60 to obtain images of desired image planes. For example, knob 96 may be utilized to rotate the transducer array 40 via drive member 60. In turn, the advancing steering, twisting and/or distal end rotation movements may be repeated. As may be appreciated, the four separate movement capabilities noted above may be utilized in various combinations, subcombinations, and ordering, thereby yielding enhanced imaging capabilities relative to known approaches.

Another embodiment of an imaging catheter 301 is shown in FIG. 9. The imaging catheter 301 may include a catheter body 310 and a distal end portion 330 supported by and selectively rotatable relative to a distal end of the catheter body 310 at an interface therebetween. The catheter body 310 may comprise a single pull wire or a plurality of pull wires and other components as described above in relation to catheter body 310. In relation to the interface between catheter body 310 and distal end portion 330, a bearing assembly 350 may be supportably disposed at the distal end of the catheter body 310 and a housing member 336 may be supportably connected to the bearing assembly 350. A transducer array 340 (e.g., an ultrasound transducer array) may be supportably interconnected to the housing member 336.

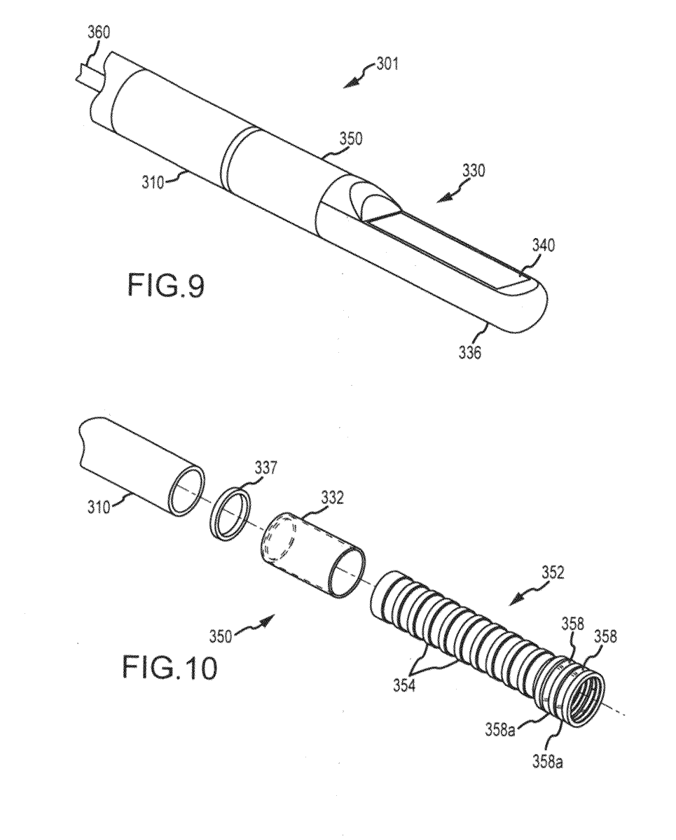

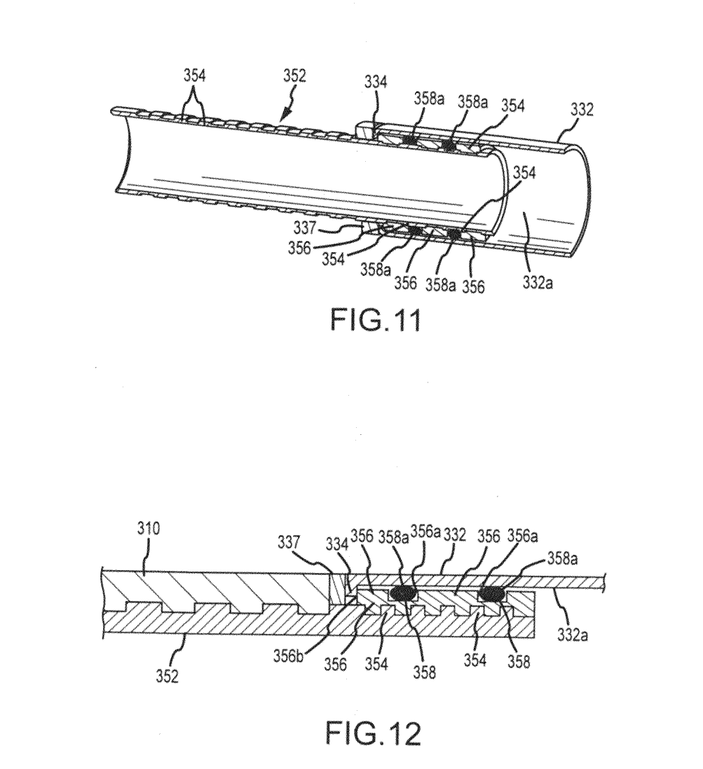

As shown in FIGS. 10-12, the bearing assembly 350 may comprise a hub 352 fixedly interconnectable to the distal end of the catheter body 310, and a coupling member 332 supportably and rotatably interconnectable to the hub 352. In turn, housing member 336 may be fixedly interconnected to the coupling member 332.

A rotatable drive member 360 may be disposed through the catheter body 310, from a proximal end to a distal end thereof, and fixedly interconnected to the housing member 336 at a distal end of the drive member 360. In turn, upon selective rotation of the proximal end of the drive member 360, the housing member 336, plug 400, transducer array 340 and coupling member 332 may be rotated. For example, transducer array 340 may be rotated to pan a predetermined imaging field across a predetermined angular range of at least 360°. In the latter regard, a bearing assembly 350 may be provided to facilitate rotation of the distal end portion 330 at least +/−180° from a “home” (e.g., center) position, and more preferably about +/−270°.

In relation to bearing assembly 350, the hub 352 may be provided to present one or more outward-facing bearing surface(s) and coupling member 332 may be provided to present one or more inward-facing bearing surface(s). Such bearing surfaces may be provided to facilitate selective rotation of the distal end portion 330 relative to catheter body 310 while maintaining contact engagement between such surfaces. In turn, such surfaces may be provided to maintain a fluid seal between the distal end portion 330 and catheter body 310, e.g., for maintaining a fluid seal at pressures up to approximately 6 psi (42 KPa). Additionally and/or alternatively, such surfaces may be provided to restrict undesired rotation of the distal end portion 330.

In the embodiment illustrated in FIGS. 9-12, the hub 352 may be provided to present outward-facing bearing surfaces 358a that are elastically deformable. In one approach, the elastically-deformable, bearing surfaces 358a may be defined by one or a plurality of spaced elastomeric O-rings 358 (e.g., spaced to enhance stability), as shown. The O-rings 358 may be disposed to be in radial compression upon assembly. In other arrangements, the O-rings or other seal means may be disposed to be in axial compression or a combination of axial and radial compression upon assembly. In various approaches, an elastically-deformable, bearing surface(s) may be defined by an elastomeric material, a thermoplastic elastomeric material, or a thermoplastic material. For example, such materials may be overmolded on hub 352 or otherwise provided by sleeves, rings, etc., positionable about hub 352.

The elastically deformable, bearing surfaces 358a may be provided to be deformably engagedly by coupling member 332 at an inward-facing, bearing surface 332a thereof. Such engagement may define a fluid seal between the distal end of the catheter body 310 and the distal end portion 330 (e.g., via spring-loading of bearing surface 358a against bearing surface 332a). Further, bearing surfaces 358a and 332a may cooperate (e.g., via frictional engagement therebetween and/or spring-loading of bearing surface 358a against bearing surface 332a) to restrict undesired rotational movement of the distal end portion 330 relative to the distal end of catheter body 310.

In various implementations, the hub 352 may include surface discontinuities 354. While the illustrated embodiment shows surface discontinuities 354 as ribs or grooves, other configurations may be utilized (e.g., knurling, ridges, through holes, etc.). The surface discontinuities 354 may be provided so that an overmolded portion 356 may flow into spaces defined between the surface discontinuities 354 during molding. In this regard, the overmolded portion 356 may be configured to define grooves 356a for restrainably receiving O-rings 358, wherein a seal is also established between the O-ring 358 and the base of grooves 356a upon assembly. The overmolded portion may also define an abutment surface 356b to facilitate retention of coupling member 332, as will be discussed.

In one embodiment, the hub 352 and coupling member 332 may each be of rigid construction comprising a metallic material, e.g., stainless steel. In turn, the overmolded portion 356 may comprise a polymeric material, thereby providing a non-metallic spacing layer between hub 352 and coupling member 332.

To assemble bearing assembly 350, coupling member 352 may be advanced over hub 352 from a proximal end of the hub 352 toward a distal end of the hub 352. The coupling member 332 may include a lip portion 334 at a proximal end of the coupling member 332. The lip portion 334 may extend radially inward from the proximal end of the coupling member 332. The lip portion 334 may be sized to abut the overmolded portion 356 at abutment surface 356b when the coupling member 332 is completely advanced from the proximal end of the hub 352 toward the distal end of the hub 352. A washer 337 may then be advanced from the proximal end of the hub 352 toward the distal end of the hub 352 until the washer 337 abuts the lip portion 334 at the proximal aspect thereof. Thereafter, the proximal end of the hub 352 may be inserted into the distal end of the catheter body 310 fixed for interconnection thereto. As may be appreciated, the washer 337 and abutment surface 356b may be provided to provide an annular slot to retain lip potion 354 of coupling member 332 therewithin, which also allows lip portion 354 to be selectively rotated therewithin. In the latter regard, the interface between lip portion 354 and abutment surface 356b at the annular slot may optionally provide a bearing interface, and in certain arrangements, may be provided to facilitate a sealed interface utilizing features taught herein.

In one embodiment, the hub 352 may be secured to the distal end of the catheter body 310 after insertion by heating the distal end of the catheter body 310 to cause polymeric material at the distal end of the catheter body 310 to flow into and at least partially fill spaces defined between the ribs 354 of the hub 352. In another embodiment, the hub 352 may be secured to the distal end of catheter body 310 via an adhesive, e.g., applied to hub 352 prior to insertion in catheter body 310 (e.g., cyanacrylate, UV-light curable adhesives, thermally-curable epoxies, etc.). In either approach, the washer 337 may be provided to present a buffer between coupling member 332 and the catheter body 310 during assembly. For example, washer 337 may be provided to have a higher melting temperature than the catheter body 310 and/or to be structurally stable in response to activation of an adhesive. Further, washer 337 is otherwise located to block material flow to bearing assembly 350 during assembly. As such, upon assembly, the bearing assembly 350 may provide for selective rotatable movement of the coupling member 332 relative to the hub 352, overmolded portion 356, and catheter body 310.

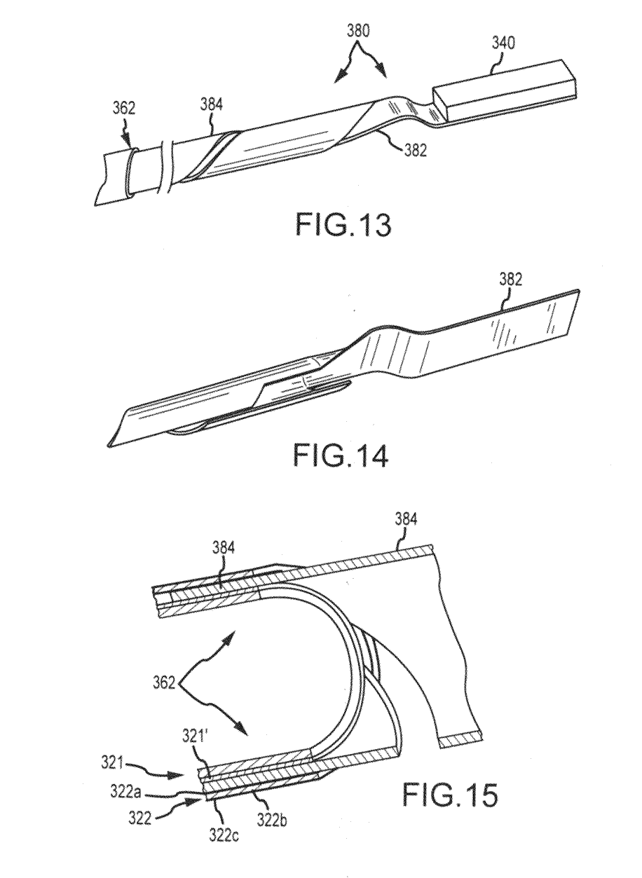

As noted, transducer array 340 may be provided for imaging a predetermined imaging field. In this regard, the imaging catheter 301 may include an electrical signal line extending between a proximal end and distal end thereof. As shown in FIG. 13, an electrical signal line 380 may include a first electrical signal member 382 electrically connected to transducer array 340, and a second electrical signal member 384 electrically connected to first electrical signal member 382. The second electrical signal member 384 may helically extend from the distal end portion 330, through the distal end of the catheter body 310 to the proximal end thereof.

The first electrical signal member 382 may comprise one or more flex board circuit members that may be flexed into a configuration as shown in FIG. 14 upon assembly of the catheter 301. As shown, such assembled configuration includes an arcuate portion that extends about and along an axis (e.g., an axis that coincides with a longitudinal axis of catheter 301, which arcuate portion electrically interfaces with a distal end of the second electrical signal member 384. In the latter regard, the second electrical signal member 384 may be of a ribbon-like configuration comprising multiple electrical lines extending along a non-conductive support layer having a conductive ground plane (e.g., 32 lines or 64 lines in certain implementations). By way of example, the second electrical signal member 384 may comprise a Microflat™ product marketed by W.L. Gore & Associates.

With reference to FIG. 15, at least a portion of the second electrical signal member 384 disposed within the catheter body 310 may be helically wound about a tubular inner member 321. Additionally, an outer tubular member 322 may be provided over the second electrical signal member 384 for at least a portion of the length of the electrical signal member 384. In one embodiment, the inner tubular member 321 may comprise an extruded polymer-based material, e.g., a polyether block amide (PEBA) such as PEBAX™. A compression layer 321′ may be provided over the inner tubular member 321. The outer tubular member 322 may include an isolation layer 322a adjacent to the second electrical signal member 384. The outer tubular member 322 may include a shielding layer 322b, e.g., comprising one or multiple conductive foils. An encapsulation layer 322c may be provided exterior to the shielding layer 322b.

In one embodiment, the assembly depicted in FIG. 15 comprising the tubular inner member 321, second electrical signal member 384, and the outer tubular layer 322, may define a drive member support structure 362. The drive member 360 may pass through the drive member support structure 362 from a proximal end to a distal end of catheter 301.

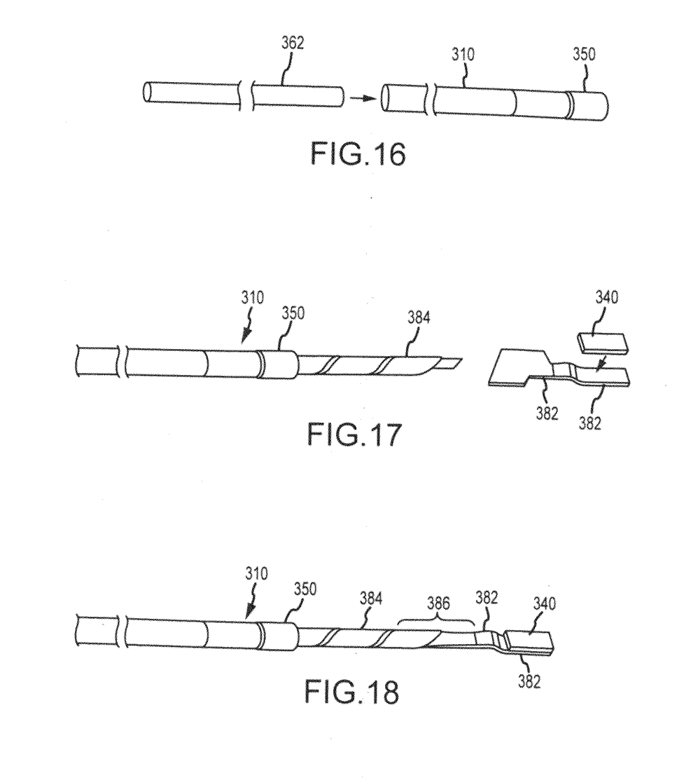

As shown in FIG. 16, to assemble the imaging catheter 301, the drive member support structure 362 may be passed through the catheter body 310 (e.g., from a proximal end to a distal end thereof). In turn, the second electrical signal member 384 may be provided to extend from the proximal end to beyond the distal end of the catheter body 310.

In one embodiment, during assembly of the catheter 301, a portion of the outer tubular member 322 shown in FIG. 15 may be removed from the drive member support structure 362. For example, the encapsulation layer 322c and shielding layer 322b may be removed at the distal end of the drive member support structure 362. The isolation layer 322a may remain in place. In one approach, the distal end of the drive member support structure 362 may be disposed in the proximal end of the catheter body 310 and advanced distally until the distal end of the drive member support structure 362 extends from the distal end of the catheter body 310 (e.g., through the bearing assembly 350).

Once the portion of the drive member support structure 362 that has been stripped of the encapsulation layer 322c and shielding layer 322b is disposed at the distal end of the catheter body 310, the isolation layer 322a may be also removed at the distal end of the drive member support structure 362. In this regard, a portion of the second electrical signal member 384 may be unwound from about the inner tubular member 321. Subsequently, the inner tubular member 321 may be removed from a distal portion of the drive member support structure 362. In turn, only an extending section, or length, of the second electrical signal member 384 may remain at the distal end of the drive member support structure 362. At least a portion of such end section may be disposed to remain free to tighten/loosen about and/or along the longitudinal axis of the catheter 301 after assembly thereof.

As shown in FIGS. 17 and 18, the first electrical signal member 382 may be operatively connected to the second electrical signal member 384 at an adjoinment region. For example, a proximal portion of first electrical signal member 382 and a distal end portion of second electrical signal member 384 may be disposed in opposing planar configurations for interconnection, wherein individual electrically conductive members provided by the second electrical signal member 384 may be disposed in electrical communication with corresponding electrically conductive members provided on the first electrical signal member 382. As the second electrical signal member 384 may be helically disposed (e.g., including the end section thereof noted above), the second electrical signal member 384 may terminate at an angle with respect to the length of the second electrical signal member 384, such that the termination of the electrically conductive members are parallel to the central axis AA of the catheter 301. For example, the second electrical signal member 384 may be cut to form the appropriate angle for termination of the electrically conductive members along the central axis AA of the catheter 301. The first electrical signal member 382 (e.g., a flex board circuit member as described above) may be correspondingly angled such that the first electrical signal member 382 provides electrical connection with the electrically conductive members parallel to a catheter axis AA.

As shown in FIG. 17, the transducer array 340 may be bonded to the first electrical signal member 384 such that electrical communication is established between the transducer array 340 and the second electrical signal member 384 by way of the first electrical signal member 382. Thus, the first electrical signal member 382 may be joined with the second electrical signal member 384 at the adjoinment region 386 shown in FIG. 18, and the transducer array 340 may be joined to the first electrical signal member 382. The adjoinment region 386 and the transducer array 340 may be disposed beyond a distal end of the catheter body during assembly to facilitate the bonding of the first and second electrical signal members 382 and 384. After electrical interconnection of the first electrical signal member 382 and second electrical signal member 384, the adjoinment region 386 may be wrapped on array housing 336, as described below.

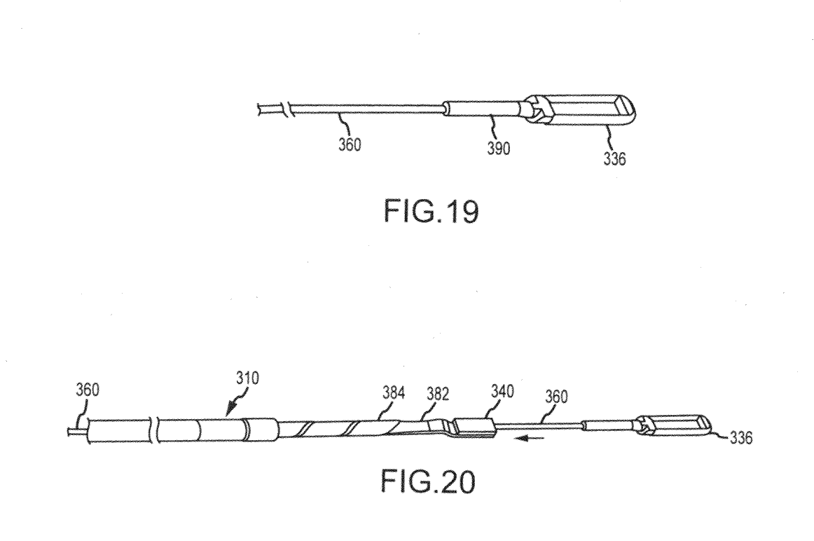

With reference to FIG. 19, the drive member 360 may be provided in a manner fixedly connected with the housing member 336 prior to assembly with other components. The housing member 336 may include a mandrel portion 390 proximal to a transducer array receiving portion 392. The housing member 336 may be secured to the drive member 360 at a distal end thereof.

With reference to FIG. 20, a proximal end of the drive member 360 may be advanced into a distal end of the catheter body 310 such that the rotatable drive member 360 is disposed within the inner tubular member 321. The rotatable drive member 360 may be advanced proximally such that the rotatable drive member 360 exits the catheter body 310 at a proximal end thereof (not shown). In this regard, as the drive member 360 is advanced proximally through the catheter body 310, the transducer array 340 may be aligned with and affixed to the transducer receiving portion 392 of housing member 336.

In one embodiment, the first electrical signal member 382 may also be affixed to a portion of the housing member 336. For example, the first electrical signal member 382 may be affixed to the housing member 336 about the mandrel portion 390. The first electrical signal member 382 may also be wound about the mandrel portion 390 and/or secured thereto (e.g., by way of a film disposed over the first electrical signal member 382, an adhesive, etc.). The first electrical signal member 382 may be provided to extend about a portion of the mandrel portion 390. For example, in some embodiments the first electrical signal member 382 may extend about approximately 360° or less a circumference of the mandrel portion 390. In one particular embodiment, the first electrical signal member 320 may extend about approximately 180° or less of the circumference of the mandrel portion 390.

As noted, a section, or length of, the second electrical signal member 384 may be provided to extend, or wind, helically from the mandrel portion 390 and into catheter body 310, free from fixed interconnection with other components between a distal end of the section (e.g., the distal end is fixedly interconnected to the first electrical signal member 382/transducer array 340/housing member 336) and a proximal end of the section (e.g., the proximal end is fixedly interconnected to the balance of the drive member support structure 362). Such section of the second electrical signal member 384 may be disposed to wind about a longitudinal axis of the catheter 301 at least one and typically a plurality of times to facilitate rotation of distal end portion 330 and steering of the catheter body 310 during use.

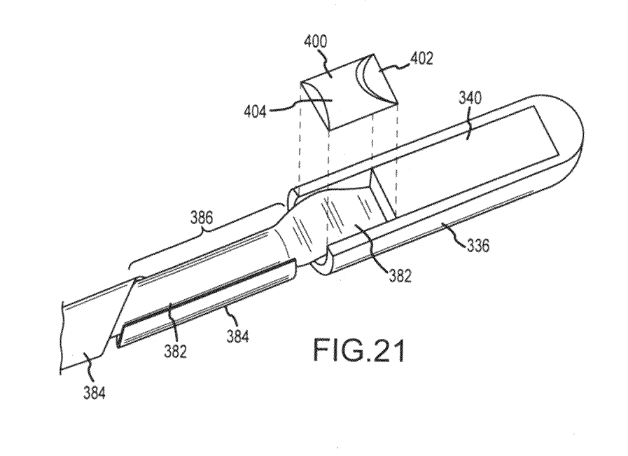

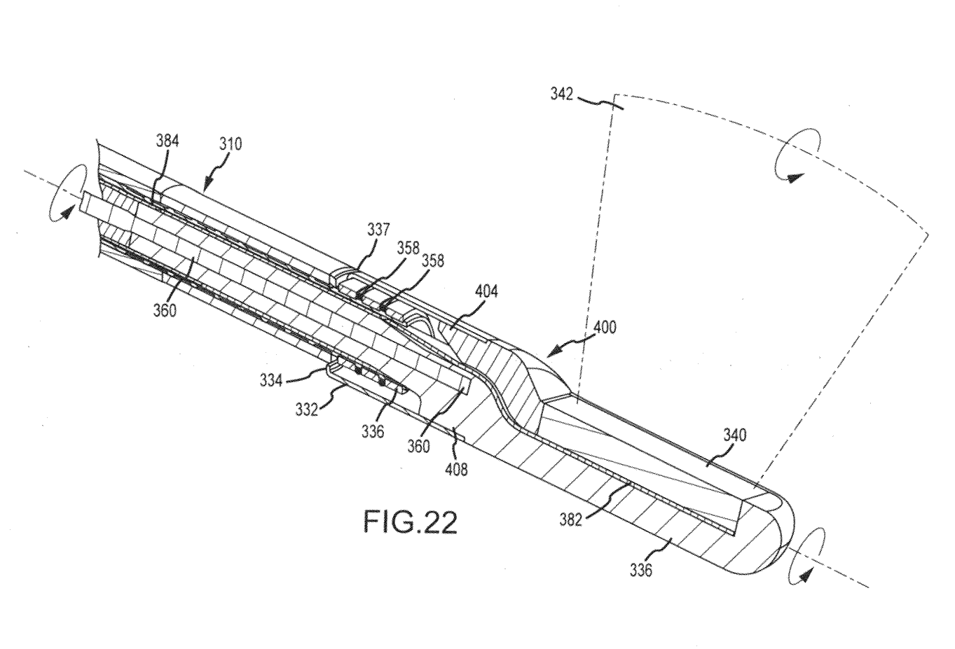

With reference to FIGS. 21 and 22, a plug 400 may be installed adjacent to the proximal end of the transducer array 340. The plug may have a transitional portion 402 and an attachment portion 404. The transitional portion 402 may provide a continuous contour from the array 340 to an outer diameter corresponding to the outer diameter of the catheter body 310. The attachment portion 404 may, along with a portion of the housing member 336 define an attachment surface 408 extending about the plug 400 and housing member 336. In this regard, the attachment surface 408 may be inserted into a distal portion of the coupling member 332 and secured thereto as shown in FIG. 21. For example, the attachment surface 408 may be secured to the coupling member 332 by way of an adhesive, a crimped portion, welding, or the like.

Prior to securing the attachment surface 408 to the coupling member 332, the second electrical signal member 384 may be wound about the drive member 360 adjacent to the distal end of the drive member 360 to facilitate insertion of the second electrical signal member 384 into the distal end of the catheter body 310 and for additional noted purposes. For example, as the second electrical signal member 384 is wound about the drive member 360, the outside diameter of the helical shape of the second electrical signal member 384 may become smaller such that the second electrical signal member 384 may be inserted to the distal end of the catheter body 310. In this regard, once the second electrical signal member 384 is able to be passed in the distal end of the catheter body 310, the housing member 336, including the attachment portion 404 of plug 400, may be advanced proximally such that the plug 400 and array housing 336 may be connected to the coupling member 332 as described above. When the array housing 336 is attached with respect to the coupling member 332, the free portion of the second electrical signal member 384 (e.g., that is the portion of the electrical signal member 384 that has been stripped of the inner tubular member 321 and outer tubular member 322 and helically disposed) may be positioned at least partially along a portion of the catheter body 310 having a relatively lower hardness than that of other portions of the catheter body 310 as described above. In this regard, the relative flexibility of the low hardness portion of the catheter body 310 may be maintained in that the second electrical signal member 384 extends freely across the portion of the catheter body 310 having relatively low hardness.

As shown in FIG. 22, rotation of the distal portion 330 may be realized by rotation of the drive member 360. Upon rotation of the distal portion 330, the coupling member 332, attached to the housing 336 and plug 400, may correspondingly rotate. As such, the lip portion 334 of the coupling member 332 may rotate with respect to the overmolded portion 356 and washer 337. In this regard, the bearing surfaces 358a and 332a may sealingly engage while allowing the relative rotation of the coupling member 332 with respect to the catheter body 310. Upon rotation of the distal portion 330, the second electrical signal member 384 may undergo corresponding rotation.

More particularly, the rotation of the distal end portion 330 may cause a winding or unwinding of the free portion of the second electrical signal member 384. That is, rotation of the distal end portion 330 in a first direction may correspond in a winding or tightening of the helical winds of the free portion of the second electrical signal member 384. Rotation of the distal end portion 330 in a second direction may result in the unwinding or loosening of the second electrical signal member 384 along the length of the free portion of the second electrical signal member 384.

In one embodiment, the distal end portion 330 may be rotatable in a plus or minus 270° range of motion. For example, as described above the free portion of the second electrical signal member 384 may be disposed adjacent to a relatively low hardness portion of the catheter body 310. Also as described above, the unwinding or loosening of the second electrical signal member 384 may correspond with an increase of the diameter of the helically wound free portion of the second electrical signal member 384. In this regard, the winding or tightening of the helically wound free portion of the second electrical signal member 384 may result in a decrease in the diameter of the free portion of the second electrical signal member 384.

The limits of the rotation of the distal portion 330 may be defined by the relative diameter of the helically wound free portion of the second electrical signal member 384. That is, when unwinding, the inner diameter of the catheter body 310 may define a first limit corresponding with a maximum outer diameter of the helically wound free portion of the second electrical signal member 384 as it abuts the inner surface of the catheter body 310. When winding, the outer diameter of the drive member 360 may define a second limit corresponding with a minimum inner diameter of the helically wound free portion of the second electrical signal member 384 as it abuts the outer surface of the drive member 360. The second limit may also be defined when winding by mechanical interference between edge portions of the second electrical signal member 384. The two limits may define the limits for rotation of the distal end portion 330. In one particular embodiment, the available degree of rotation of the distal end portion 330 may be established to be at least +/−270°.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain known modes of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. An imaging catheter comprising:
a catheter body having a proximal end and a distal end;
a distal end portion supported by and selectively rotatable relative to the distal end of the catheter body at an interface therebetween;
at least one electrical signal line extending within said catheter body between said proximal end and said distal end thereof; and,
a transducer array supported by said distal end portion and electrically interconnected to said electrical signal line across said interface, wherein said transducer array has a predetermined imaging field that is selectively rotatable about an axis extending distally away from the distal end of the catheter body.

2. The imaging catheter of claim 1, wherein said interface provides a fluid seal between said distal end of said catheter body and said rotatable distal end portion.

3. The imaging catheter of claim 1, wherein said interface comprises:
a first bearing surface at said distal end of said catheter body; and,
a second bearing surface on said distal end portion, wherein said first bearing surface and said second bearing surface are disposed in opposing contact relation.

4. The imaging catheter of claim 3, wherein said first bearing surface and said second bearing surface cooperate to provide a fluid seal between said distal end of said catheter body and said distal end portion.

5. The image catheter of claim 4, wherein one of said first bearing surface and said second bearing surface is elastically deformed.

6. The imaging catheter of claim 5, wherein one of said first bearing surface and said second bearing surface comprises one of an elastomeric material, a thermoplastic material, and a thermoplastic elastomeric material.

7. The imaging catheter of claim 4, wherein opposing portions of said first bearing surface and said second bearing surface having coincidental configurations extending about a longitudinal axis of the imaging catheter.

8. The imaging catheter of claim 4, wherein opposing portions of said first bearing surface and said second bearing surface having annular configurations extending about a longitudinal axis of the imaging catheter.

9. The imaging catheter of claim 8, wherein substantially all contact between said first bearing surface and said second bearing surface along said longitudinal axis is one of equidistance from said longitudinal axis.

10. The image catheter of claim 2, wherein said interface comprises:
a plurality of first bearing surfaces at said distal end of said catheter body, wherein said plurality of first bearing surfaces spaced along a longitudinal axis of the imaging catheter; and
at least one second bearing surface on said distal end portion, wherein said plurality of first bearing surfaces and said at least one second bearing surface are disposed in opposing contact relation to provide a fluid seal between said distal end of said catheter body and said distal end portion.

11. The imaging catheter of claim 10, wherein one of said plurality of first bearing surfaces and said at least one second bearing surface is elastically deformed.

12. The imaging catheter of claim 11, wherein one of said plurality of first bearing surfaces and said at least one second bearing surface comprises an elastomeric material.

13. The imaging catheter of claim 10, wherein opposing portions of said plurality of first bearing surfaces and said at least one second bearing surface have coincidental configurations extending about a longitudinal axis of the imaging catheter.

14. The imaging catheter of claim 10, wherein opposing portions of said plurality of first bearing surfaces and said at least one second bearing surface having annular configurations extending about a longitudinal axis of the imaging catheter.

15. The imaging catheter of claim 14, wherein substantially all contact between said plurality of first bearing surfaces and said at least one second bearing surface along said longitudinal axis is equidistance from said longitudinal axis.

16. The imaging catheter of claim 1, further comprising:
a force communication member operable to selectively applying a force to rotate said distal end portion relative to said distal end of said catheter body.

17. The imaging catheter of claim 16, wherein said interface restricts rotative movement of said distal end portion relative to said distal end of said catheter body in the absence of application of said force.

18. The imaging catheter of claim 17, wherein said interface comprises:
a first bearing surface at said distal end of said catheter body; and,
a second bearing surface on said distal end portion.

19. The imaging catheter of claim 18, wherein said first bearing surface and said second bearing surface cooperate to restrict rotative movement of said distal end portion relative to said distal end of said catheter body in the absence of application of said force.

20. The imaging catheter of claim 19, wherein one of said first bearing surface and said second bearing surface is elastically deformed.

21. The imaging catheter of claim 20, wherein one of said first bearing surface and said second bearing surface comprises one of an elastomeric material, a thermoplastic material, and a thermoplastic elastomeric material.

22. The imaging catheter of claim 18, wherein opposing portions of said first bearing surface and said second bearing surface having coincidental configurations extending about a longitudinal axis of the imaging catheter.

23. The imaging catheter of claim 16, wherein opposing portions of said first bearing surface and said second bearing surface having annular configurations extending about a longitudinal axis of the imaging catheter.

24. The imaging catheter of claim 20, wherein substantially all contact between said first bearing surface and said second bearing surface along said longitudinal axis is equidistance from said longitudinal axis.

25. The imaging catheter of claim 17, wherein said interface comprises:
a plurality of first bearing surfaces at said distal end of said catheter body, wherein said plurality of first bearing surfaces spaced along a longitudinal axis of the imaging catheter; and
at least one second bearing surface on said distal end portion, wherein said plurality of first bearing surfaces and said at least one second bearing surface cooperate to restrict rotative movement of said distal end portion relative to said distal end of said catheter body in the absence of application of said force.

26. The imaging catheter of claim 25, wherein one of said plurality of first bearing surfaces and at least one second bearing surface is elastically deformed.

27. The imaging catheter of claim 25, wherein opposing portions of said plurality of first bearing surfaces and at least one second bearing surface have coincidental configurations extending about a longitudinal axis of the imaging catheter.

28. The imaging catheter of claim 25, wherein opposing portions of said plurality of first bearing surfaces and said at least one second bearing surface having annular configurations extending about a longitudinal axis of the imaging catheter.

29. The imaging catheter of claim 25, wherein substantially all contact between said plurality of first bearing surfaces and said at least one second bearing surface along said longitudinal axis is equidistance from said longitudinal axis.

30. The imaging catheter of claim 16, wherein said force communication member comprises:
a drive member extending through said catheter body from said proximal end to said distal end thereof, and interconnected to said distal end portion, wherein a proximal end of the drive member is selectively rotatable to affect said selective rotation of said distal end portion and said predetermined imaging field.

31. The imaging catheter of claim 30, wherein said drive member comprises a tubular member, and wherein at least a portion of said electrical signal line extends through the tubular member.

32. The imaging catheter of claim 30, wherein said catheter body includes a tubular passageway extending from said proximal end to said distal end thereof, wherein said drive member extends through said passageway from said proximal end to said distal end thereof.

33. The imaging catheter of claim 1, wherein said catheter comprises:
at least a first segment having a first stiffness;
a second segment disposed distal to the first segment and having a second stiffness, wherein the first stiffness is greater than the second stiffness; and,
at least one pull wire extending from said proximal end of the catheter body through corresponding a passageway extending through at least a portion of said first segment and at least a portion of said second segment to an anchor location offset from a center axis of the catheter body, wherein said second segment is deformable to a smaller radius of curative than that of said first segment in response to a tensile force applied to said at least one pull wire.

34. The imaging catheter of claim 33, wherein said first segment is deformable to a first radius of curvature (R₁) and said second segment is deformable to a second radius of curative (R₂) in response to a tensile force applied to said at least one pull wire, and wherein a ratio of R₂/R₁ is no more than about 2/3.

35. The imaging catheter of claim 33, wherein said second segment comprises said distal end of said catheter body, and wherein said second segment to deformable to a second radius of curvature of about 4 cm. or less.

36. The imaging catheter of claim 33, wherein said catheter is deformable to a predetermined minimum radius of curvature along an entire length thereof in response to a tensile force applied to said at least one pull wire, wherein said at least one electrical signal line is of a ribbon-like configuration and extends helically about said center axis of the catheter body within a predetermined wrap angle range established so that said at least one electrical signal line maintains a non-overlapping disposition when said catheter body is deformed to said predetermined minimum radius of curvature.

37. The imaging catheter of claim 1, wherein said at least one electrical signal line comprises:
a first electrical signal member of a ribbon-like configuration that extends helically about a center axis of the catheter body; and
a second electrical signal member including at least one flex board circuit member electrically interconnected to and extending distally from a distal end of said first electrical signal member, wherein a length of said first electrical signal member coils upon rotation of said distal end portion of the imaging catheter in a first direction and uncoils upon rotation of said distal end portion of the imaging catheter in a second direction, opposite to said first direction.

38. The imaging catheter of claim 37, wherein said at least one flex board circuit member is arcuately flexed about and along said center axis of the imaging catheter.

39. The imaging catheter of claim 37, wherein said first electrical signal member extends helically from said catheter body in to the distal end portion, and wherein said at least one flex board is located within the distal end portion.

40. The imaging catheter of claim 37, said distal end portion comprising:
a housing member for supporting said transducer array;
a drive member extending through said catheter body from said proximal end to said distal end thereof, and interconnected to said housing member, wherein a proximal end of the drive member is selectively rotatable to affect said selective rotation of said housing member and said predetermined imaging field.

41. A method for imaging a predetermined region of interest within a patient body, comprising:
advancing a catheter in the patient body, wherein the catheter includes a catheter body and a distal end portion supported thereby;
rotating said distal end portion relative to a distal end of the catheter body;
obtaining an imaging signal from a transducer array supported by said distal end after at least a portion of said rotating step, wherein said transducer array has a predetermined imaging field that is positionable at a plurality of locations; and,
processing said imaging signal to obtain image data corresponding with the plurality of locations of the predetermined imaging field.

42. A method as recited in claim 41, wherein said distal end portion and said catheter body of said catheter comprise a first bearing surface and a second bearing surface, respectively, and wherein said rotating step comprises:
moving said first bearing surface relative to and in contact engagement with said second bearing surface.

43. A method as recited in claim 42, further comprising:
maintaining a fluid seal between said distal end portion and a distal end of said catheter body throughout said moving step.

44. A method as recited in claim 42, wherein said first bearing surface and said second bearing surface are provided to resist relative movement therebetween in the absence of the application of a predetermined minimum force to said distal end portion.

45. A method as recited in claim 44, wherein said catheter includes a drive member interconnected to the distal end portion and extending from a proximal end of the catheter body to a distal end thereof, wherein said rotating step further comprises:
manipulating said drive member at said proximal end of the catheter body to apply at least said predetermined amount of force to affect selective rotation of said distal end portion.

46. A method as recited in claim 41, further comprising:
steering said catheter to curve the catheter body along a length thereof and thereby position the distal end portion.

47. A method as recited in claim 46, wherein said steering and said rotating steps are completed free from advancement of the catheter.

48. A method as recited in claim 46, further comprising:
twisting the catheter to rotate the catheter body and distal end portion.

49. The imaging catheter of claim 37, wherein a distal end portion of the first electrical signal member terminates at an angle along a length thereof.

50. The imaging catheter of claim 49, wherein said second electrical signal member is angled in corresponding relation to said distal end portion of the first electrical signal member for electrical connection therewith.

51. The imaging catheter of claim 50, wherein said second electrical signal member includes an arcuately flexed portion that extends about and along said center axis of said catheter body to electrically interface with the distal end portion of the first electrical signal member.

52. The imaging catheter of claim 50, wherein said first electrical signal member comprises a plurality of electrically conductive members and said second electrical signal member comprises a plurality of electrically conductive members, and wherein individual ones of said plurality of electrically conductive members of said first electrical signal member are disposed in electrical communication with individual ones of said plurality of electrically conductive members of said second electrical signal member.

53. The imaging catheter of claim 52, wherein termination of the plurality of electrically conductive members of the first electrical signal line at the distal end portion thereof is along the center axis of the catheter body so that said plurality of electrically conductive members of the first electrical signal member and the second electrical signal member, respectively, are electrically connected parallel to the center axis of the catheter body.
